(19) United States
(12) Patent Application Publication
(10) Pub. No.: US 2004/0013720 A1
(43) Pub. Date: Jan. 22, 2004

(54) Receptor antagonist-lipid conjugates and delivery vehicles containing same

(76) Inventors: Harma M. Ellens, King of Prussia, PA (US); Myrna A. Monck, Collegeville, PA (US); Ping-Yang Yeh, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/415,160

(22) PCT Filed:
(86) PCT No.: PCT/US01/46206

(51) Int. Cl. A61K 9/127

(52) U.S. Cl. 424/450

(57) ABSTRACT

Disclosed are vesicular drug delivery vehicles, such as liposomes, comprising a targeting ligand which comprises a non-biological, biomitric antagonist to a receptor that is upregulated at a disease site.

### FIELD OF THE INVENTION

The present invention relates to vesicular drug delivery vehicles, such as liposomes, comprising a targeting ligand which comprises a non-biological, biomimetic antagonist to a receptor that is upregulated at a disease site.

### BACKGROUND OF THE INVENTION

Liposomes, spherical vesicles comprising one or more lipid bilayers comprising amphipathic, vesicle-forming lipids, are employed for in vivo administration of a variety of therapeutic agents. Liposomal dosage forms are particularly useful for delivering therapeutics tending to have toxic side effects, such as anti-cancer drugs. Particularly commercially useful liposomes are long circulating liposomes that avoid uptake by the mononuclear phagocyte system. An example of such liposomes are those comprising hydrophilic polymer on the liposome surface, such as STEALTH® liposomes.

Approaches have been taken to provide site-specific delivery of liposomes. In such approaches, a targeting ligand may be attached to the liposome surface, typically by coupling to a lipid comprising the liposomal bilayer. Targeting ligands have typically included antibodies, antibody fragments, peptides and other biological materials such as certain vitamins and sugars, especially antibodies and antibody fragments.

However, the use of such biological ligands has certain disadvantages. Antibodies and antibody fragments are susceptible to degradation, presenting liposome shelf life, manufacturing, and integrity concerns. In particular, it is generally necessary to specially handle these biological materials to minimize degradation. Therefore, liposomes comprising targeting antibodies or antibody fragments typically couple the antibody material to the exterior liposome surface only after preparation of the liposome, thereby requiring an additional manufacturing step. In addition, this post-insertion of the ligand can cause liposome bilayer defects resulting in vesicle leakage, reducing acceptable product yield or causing administration of the therapeutic agent to be less controlled. Furthermore, antibody materials can potentially suffer from immunogenicity issues.

Peptide ligands suffer from other problems. For instance, peptides often require special chemical processing in order to control the coupling reaction of the peptide and the lipids at the liposome surface. For example, peptides may comprise several free acidic, amino and/or sulfhydryl groups which are capable of reacting with the lipids. Protection of amino acid side chains may be required during insertion of the peptide ligand into the liposome, followed by deprotection steps, or other chemistries may be required to avoid cross-reactions. In addition, peptides tend to be costly, and like antibodies can be immunogenic and susceptible to degradation, requiring special handling.

Other biological ligand materials which have been described, such as certain vitamins and sugars, can suffer from similar issues of immunogenicity and the need to chemically manage multiple functional groups.

It would be desirable to provide a targeted liposome which can be produced cost-effectively and reliably on a commercial scale, in order to make treatment with liposomal therapeutics more accessible to patients. In particular it is desirable to provide targeted liposomes which have good shelf stability and integrity and which are manufactured by relatively simple processes. For example, it is desirable to provide a liposome that can be targeted by insertion of a targeting ligand during preparation of the liposome, which involves relatively straightforward manufacturing processes. Furthermore, it is desirable to provide targeted liposomes which do not present significant immunogenic potential and which have good binding affinity to the target delivery site.

It is also known that certain receptors, including integrins such as the vitronectin (αᵥβ₃) receptor, are upregulated on the surface of growing endothelial cells. It is also known that the progression of a cancerous tumor involves processes characterized by neovascularization (angiogenesis), more particularly that angiogenesis is a crucial step in a tumor's transition from a small cluster of mutated cells to a malignant growth. It is also known that inhibition of this angiogenesis will limit tumor progression and formation and progression of metastases. On this basis, anti-angiogenic agents have been proposed for the treatment of cancer. For example, a peptide-drug conjugate that binds to the αᵥβ₃ and αᵥβ₅ receptors has been shown to be a very potent anti-angiogenic compound, as blocking the αᵥβ₃ or αᵥβ₅ receptors results in the death of proliferating endothelial cells. Pasqualini, R. et al., Nature Biotechnology, Vol. 15, pp. 542-546 (1997).

Non-peptide receptor antagonists selective for one or more integrins, such as the vitronectin receptor (αᵥβ₃) and αᵥβ₅ receptor, are also known. See, e.g., Nicolau, K. C. et al., Design, Synthesis and Biological Evaluation of Nonpeptide Integrin antagonists, Bioorganic & Medicinal Chemistry 6 (1998) 1185-1208. Recent PCT publications disclose pharmaceutically active compounds which inhibit the vitronectin receptor and which are useful for the treatment of inflammation, cancer, cardiovascular disorders, such as atherosclerosis and restenosis, and/or diseases wherein bone resorption is a factor, such as osteoporosis, including: PCT applications WO 96/00730, published Jan. 11, 1996; WO 97/24119, published Jul. 10, 1992; WO 98/14192, published Apr. 9, 1998; WO98/30542, published Jul. 16, 1998; WO99/15508, published Apr. 1, 1999; WO99/05232, published Sep. 16, 1999; WO00/33838, published Jun. 15, 2000; WO97/01540, published Jan. 16, 1997; WO99/15170, published Apr. 1, 1999; WO99/15178, published Apr. 1, 1999; WO00/07544, published Feb. 17, 2000; WO96/00574, published Jan. 11, 1996; WO97/24122, published Jul. 10, 1997; WO97/24124, published Jul. 10, 1997; and WO99/05107, published Feb. 4, 1999. Inhibitors of the vitronectin receptor are also disclosed in WO 00/35887, published Jun. 22, 2000.

The present invention involves the discovery that therapeutic liposomes can be targeted to disease sites through non-biological, biomimetic ligands incorporated into the liposome. Such liposomes comprising non-biological, biomimetic targeting ligands can be manufactured more economically and reliably on a commercial scale relative to processes typically required to manufacture liposomes comprising various biological ligands, and possess good shelf life, integrity, and relatively low immunogenic potential.

The present invention also involves the discovery that diseases characterized by angiogenesis can be effectively treated or diagnosed by administration of liposomes comprising a non-biological, biomimetic antagonist to receptors upregulated on the surface of growing endothelial cells present at the disease site, e.g., the αᵥβ₃ or αᵥβ₅ receptor.

### SUMMARY OF THE INVENTION

The present invention relates to liposomes having a conjugate bound to its lipid bilayer, wherein the conjugate comprises (a) a vesicle-forming lipid having a polar head group and a hydrophobic tail, and (b) a non-biological, biomimetic antagonist to a receptor upregulated at a disease site, directly or indirectly chemically linked to the polar head group of the vesicle-forming lipid.

The antagonist preferably binds a receptor upregulated in the vascular endothelium of inflammation, infection or tumor sites, and is more preferably an integrin receptor antagonist, most preferably a vitronectin receptor antagonist.

The conjugate preferably further comprises a hydrophilic polymer having a proximal end and a distal end, wherein the polymer is chemically linked at its proximal end to the polar head group of the vesicle-forming lipid conjugate and chemically linked at its distal end to the antagonist. Polyalkylethers, e.g., polyoxyethylene glycol, and alkoxy-capped analogs thereof are preferred hydrophilic polymers.

The liposomes preferably comprise a therapeutic or diagnostic active agent, more preferably selected from anti-neoplastic agents, anti-inflammatory agents, anti-infective agents, diagnostic imaging agents and combinations thereof. The invention is particularly well suited for administration of anti-neoplastic agents such as camptothecins and especially topotecan.

The conjugate is advantageously inserted into the liposome during preparation of the liposome. The conjugate may alternatively be inserted into pre-formed liposomes. In either embodiment, the conjugate may be pre-formed or may be formed in situ.

The present invention also relates to the conjugate.

The invention also relates to a method of treating or diagnosing a disease characterized by upregulation of a receptor, comprising administering to a patient in need thereof a safe and effective amount of such liposomes, wherein the antagonist has binding affinity to the upregulated receptor. In a preferred embodiment the receptor is upregulated in the vascular endothelium of inflammation, infection or tumor sites and the disease is characterized by angiogenesis, such as osteo arthritis, rhumatoid arthritis, diabetic retinopathy, hemangiomas, psoriasis, restenosis or a cancerous tumor. A preferred receptor is an integrin, more preferably the vitronectin receptor, and a preferred antagonist is an integrin- and especially a vitronectin receptor-antagonist.

The invention also relates to pharmaceutical compositions comprising such liposomes and a pharmaceutically acceptable carrier or diluent.

### DETAILED DESCRIPTION

All documents cited or referred to herein, including issued patents, published and unpublished patent applications, and other publications are hereby incorporated herein by reference as though fully set forth.

Certain components of the present invention, such as lipids and active agents, are grouped herein according to certain classifications. It will be recognized that components may belong to one or more classes, therefore their listing in a particular class is not intended to be limiting.

Preferred drug delivery vehicles of the present invention are liposomes, including unilamellar and multilamellar liposomes. Unilamellar, or single lamellar liposomes, are spherical vesicles comprising a lipid bilayer membrane that defines a closed compartment. The bilayer membrane is composed of two layers of lipids: an outer layer of lipid molecules with the hydrophilic head portions thereof oriented toward the external aqueous environment and the hydrophobic tails thereof oriented toward the interior of the liposome; and an inner layer laying directly beneath the outer layer wherein the lipid molecules are oriented with the heads toward the aqueous interior of the liposome and the tails toward the tails of the outer lipid layer. Multilamellar liposomes are spherical vesicles that comprise more than one lipid bilayer membrane which define more than one closed compartment. The membranes are concentrically arranged so that they are separated by compartments much like an onion.

The liposomes comprise one or more vesicle-forming lipid materials such as are known in the art, preferably having two hydrocarbon chains (e.g., acyl chains), and a polar or non-polar headgroup, typically polar. Suitable vesicle-forming lipids may be selected from the group consisting of:

(1) phospholipids, such as:

(a) phosphatidylcholines [PC] (e.g., L-α-dipalmitoylphosphatidylcholine [DPPC], L-α-dimyristoylphosphatidylcholine [DMPC]), 1-palmitoyl-2-oleoylphosphatidylcholine [POPC], hydrogenated soy phosphatidylcholine [HSPC], and L-α-distearoylphosphatidylcholine [DSPC]);

(b) phosphatidylglycerols (e.g., L-α-dimyristoylphosphatidylglycerol);

(c) phosphatidyl-ethanolamines [PE] (e.g., distearylphosphatidylethanoloamine [DSPE], dimyristoylphosphatidylethanolamine [DMPE]);

(d) phosphatidylinositols [PI];

(e) phosphatidic acids [PA]; and

(f) phosphatidylserines;

(2) sterols (such as cholesterol and related sterols);

(3) glycolipids (such as cerebroside, gangliosides);

(4) cationic lipids (such as gemini surfactants, including those disclosed in WO 99/29712, published June 17, 1999, Patrick Camilleri et al.);

(5) sphingolipids (such sphingomyelin [SM] and ceramides);

(6) glycerolipids (such as neutral or non-neutral diacylglycerols, triacylglycerols); and

(7) hydrophilic polymer—derivatives of any of the foregoing lipids (e.g., such as described below)

The vesicle-forming lipids may be selected by the skilled artisan according to known principles, for example to provide liposomes having more or less rigidity, fluidity, permeability, mechanical strength, blood circulation half-life, serum-stability and the like.

In a preferred embodiment, the liposomes comprise at least one vesicle-forming lipid that is derivatized with a hydrophilic polymer, more preferably a non-antigenic, hydrophilic polymer. Liposomes comprising the hydrophilic polymers have increased blood circulation time, and therefore tend to provide improved delivery of the liposome to the targeted site, relative to liposomes not containing such polymers.

Suitable hydrophilic polymers include synthetic and natural polymers. Synthetic polymers include homopolymers and block or random copolymers. Suitable hydrophilic synthetic polymers include polyalkyl (e.g., C1-4) ethers and alkoxy (e.g., C1-4)-capped analogs thereof; polyvinylpyrrolidone; polyvinylalkyl (e.g., C1-4 such as methyl) ether; polyalkyl (e.g., C1-4 such as methyl, ethyl, propyl) oxazoline; polyhydroxyalkyl (e.g., C1-4 such as methyl, ethyl, propyl) oxazoline; polyalkyl (e.g., C1-4 such as meth-, dimeth-) acrylamide; polyhydroxyalkyl (e.g., C1-4 such as propylmeth-) acrylamide; polyhydroxyalkyl (e.g., C1-4 such as ethyl-, propylmeth-) acrylate;hydroxyalkyl (e.g. C1-4 such as methyl-, ethyl-) cellulose. Natural hydrophilic polymers include polysialic acids and analogs thereof, polyaspartamide and hydrophilic peptide sequences. For example, the use of polysialic acids is described in U.S. Pat. No. 5,846,951 issued to Gregory Gregoriadis on Dec. 8, 1998.

Preferred are polyalkylethers and alkoxy-capped analogs thereof, such as polyoxyethylene glycol, polyoxypropylene glycol, polyoxyethylene/propylene glycol, and methoxy or ethoxy—capped analogs thereof. Polyoxyethylene glycol is more preferred, even more preferably having a molecular weight of about 300-7000.

Suitable hydrophilic polymers, their preparation and use in liposomes are described, for example, in U.S. Pat. No. 5,013,556 issued to Woodle et al. on May 7, 1991 and U.S. Pat. No. 5,395,619. Liposomes comprising such hydrophilic polymers are well known in the art and include those known as sterically stabilized or STEALTH® liposomes. See, e.g., Lasic, D. D., Recent Developments in Medical Applications of Liposomes: Sterically Stabilized Liposomes in Cancer Therapy and Gene Delivery In Vivo, J. Control Release, Vol 48, Issue 2-3, pp. 203-222 (1997). Long circulating liposomes and components thereof suitable for use in the present invention are also described in Papahadjopoulos D. et al., (1991): Sterically stabilized liposomes: improvements in pharmacokinetics and antitumor efficacy. Proc Natl Acad Sci USA 88:11460-11464; Gabizon A. et al., (1988): Liposome formulations with prolonged circulation time in blood and enhance uptake by tumors. Proc Natl Acad Sci USA 85:6949-6953; Huang S. K. et al. (1992): Pharmacokinetics and therapeutics of sterically stabilized liposomes in mice bearing C-26 colon carcinoma. Cancer Research 52:6774-6781; Webb M. S. et al (1995): Sphingomyelin-cholesterol liposomes significantly enhance the pharmacokinetic and therapeutic properties of vincristine in murine and human tumour models. British Journal of Cancer 72:895-904; Northfelt D. W. et al. (1996): Doxorubicin encapsulated in liposomes containing surface-bound polyethylene glycol: pharmacokinetics, tumor localization, and safety in patients with AIDS-related Kaposi's Sarcoma. J. Clin. Pharmacol. 36:55-63; Gill P. S. et al. (1995): Phase I/II clinical and pharmacokinetic evaluation of liposomal daunorubicin. Journal of clinical Oncology 13:996-1003.

In preferred embodiments, the liposome comprises a lipid material selected from the group consisting of HSPC, DSPC, DPPC, DMPC, POPC, sphingomyelin, EggPC, optionally cholesterol, and optionally a PEGylated lipid such as PEGylated DSPE or PEGylated DMPE.

The drug delivery vehicles of the present invention comprise one or more antagonists to a receptor upregulated at a disease site. The antagonist is an organic molecule which can bind the receptor. The antagonists are non-biological, being synthetic material not isolated or derived from a biological source. Thus the present invention excludes peptides, antibodies, antibody fragments, vitamins and sugars, which are isolated or derived from biological sources. The antagonists are biomimetic, in that they bind a receptor.

Preferred antagonists have a high degree of selectivity and a high binding affinity to a receptor of interest. Suitable antagonists comprise a functional group for coupling to the lipid, and if used, optionally the hydrophilic polymer and/or other linking moieties in forming the conjugates described herein. The antagonist can therefore be described as comprising a receptor antagonist template, which as used herein refers to the core structure of an antagonist to a receptor upregulated at a disease site, which core is substituted by a functional group for coupling to the lipid, and if used, optionally the hydrophilic polymer and/or other linking moieties in forming the conjugates described herein.

Suitable non-biological, biomimetic antagonists for use in the present invention include those that bind to a receptor that is upregulated in the vascular endothelium of inflammation, infection or tumor sites. Examples of receptors that are upregulated in the vascular endothelium of inflammation, infection or tumor sites are integrin receptors, such as αVβ3, αVβ5 and α5β1 Prostate Specific Membrane Antigen (PSMA) receptor, Herceptin, Tie1 receptor, Tie2 receptor, ICAM1, Folate receptor, basic Fibroblast Growth Factor (bFGF) receptor, Epidermal Growth Factor (EGF) receptor, Vascular Endothelial Growth Factor (VEGF), Platelet Derived Growth Factor (PDGF) receptor, Laminin receptor, Endoglin, Vascular Cell Adhesion Molecule VCAM-1, E-Selectin, and P-Selectin.

Suitable non-biological, biomimetic antagonists include:

(1) Analogs of YIGSR—NH2 (peptidomimetic inhibitors of the laminin receptor, such as described in Zhao M., Kleinman H K., and Mokotoff M., Synthesis and Activity of Partial Retro-Inverso Analogs of the Antimetastatic Laminin-Derived Peptide, YIGSR-NH2. International Journal of Peptide & Protein Research. 49(3):240-253, March 1997

(2) PD156707 and derivatives thereof (such as described in Harland S P., Kuc R E., Pickard J D., Davenport A P. Expression of Endothelin(A) Receptors in Human Gliomas and Meningiomas, with High Affinity for the Selective Antagonist PD156707. Neurosurgery. 43(4):890-898, October 1998.

(3) Integrin receptor antagonists, including antagonists to the receptors αVβ3 (vitronectin receptor), αVβ5 and αVβ1

Suitable antagonists are those which comprise a functional group for linking to the lipid or optional hydrophilic polymer or linking moiety to form the conjugate as described above, or which comprise a receptor antagonist template and which can be derivatized by known methods to comprise such a functional group. Integrin receptor antagonists are preferred, antagonists to the receptors αVβ3, αVβ5 and α5β1, and especially αVβ3 being more preferred. Such antagonists will be RGD mimetics, and will comprise a functional group for coupling to the lipid, and if used, optionally the hydrophilic polymer and/or other linking moieties in forming the conjugates described herein. Preferred functional groups are primary aliphatic (e.g., C3-C18) amines, carboxylic acids, sulfates or sulfhydryls, more preferably amines or carboxylic acids. RGD mimetics having such functional groups are known in the art, or are readily prepared from known RGD mimetics using conventional synthetic chemistry. As will be understood by those skilled in the art, incorporation of such functional groups will be designed so as to substantially retain the RGD mimetic character of the parent compound.

For example, RGD mimetics which can be adapted for use in the present invention may be selected from the integrin receptor antagonists described in Nicolau, K. C. et al., Design, Synthesis and Biological Evaluation of Nonpeptide Integrin Antagonists, Bioorganic & Medicinal Chemistry 6 (1998) 1185-1208, and in PCT applications WO 96/00730, published Jan. 11, 1996; WO 97/24119, published Jul. 10, 1992; WO 98/14192, published Apr. 9, 1998; WO98/30542, published Jul. 16, 1998; WO99/15508, published Apr. 1, 1999; WO99/05232, published Sep. 16, 1999; WO00/33838, published Jun. 15, 2000; WO97/01540, published Jan. 16, 1997; WO99/15170, published Apr. 1, 1999; WO99/15178, published Apr. 1, 1999; WO00/07544, published Feb. 17, 2000; WO96/00574, published Jan. 11, 1996; WO97/24122, published Jul. 10, 1997; WO97/24124, published Jul. 10, 1997; WO99/05107, published Feb. 4, 1999; PCT application No. PCT/US00/24514, filed Sep. 7, 2000; WO 00/35887, published Jun. 22, 2000; U.S. Pat. No. 5,929,120; and W. H. Miller et al., Indentification and in vivo Efficacy of Small-Molecule Antagonists of Integrin αVβ3 (the Vitronectin Receptor), Drug Discovery Today, Vol. 5, Issue 9, Sept. 1, 2000, pp 397-408.

Examples of vitronectin receptor antagonists (“VRAs”) include compounds represented by the following structures:

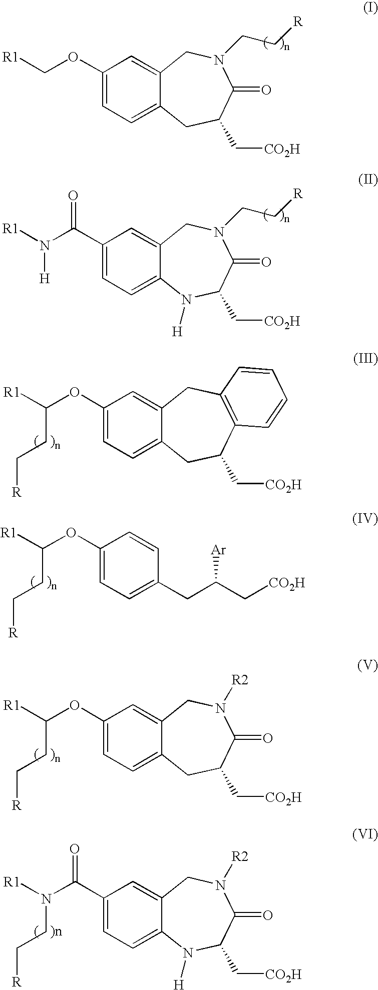

wherein the above structures (I)-(VI):

R is selected from NH₂, COOH, and SH

R1 is selected from:

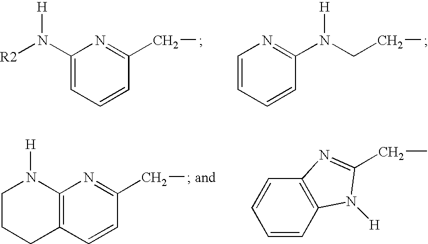

R2 is H or 1-4 C alkyl, especially H or CH3, and

n is an integer from 0-20, especially 0-5, e.g., 1-5.

In a preferred embodiment the vitronectin receptor antagonist has the structure:

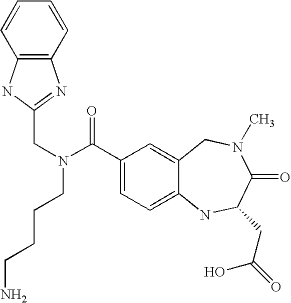

In another embodiment, the antagonist is the amino derivative of the structure:

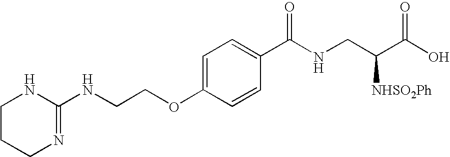

This compound and its synthesis is described in U.S. Pat. No. 5,929,120. The amino derivative can be prepared by one skilled in the art by substituting the phenyl sulfonyl with hydrogen.

In a preferred embodiment the antagonist is chemically linked, preferably covalently linked, to a lipid material having a polar head group and a hydrophobic tail to form a receptor antagonist-lipid conjugate. In a preferred embodiment the conjugate comprises the lipid material, a hydrophilic polymer chemically linked, preferably covalently, to the polar head group of the lipid, and the antagonist which is chemically linked, preferably covalently, to the hydrophilic polymer. The conjugates are novel compounds and are useful as intermediates in preparing the liposomes of the invention. The conjugates therefore comprise part of the present invention.

Suitable lipids for forming the conjugate include the vesicle-forming lipid materials described above, which comprise or are readily derivatized to comprise a functional group for coupling to the receptor antagonist and, if used in the conjugate, the hydrophilic polymer or other linking moieties described herein. Vesicle-forming lipids used in the conjugates are preferably selected from gemini surfactants, phosphatidylethanolamines, phosphatidylserines, other glycerolipids, and sphingolipids (e.g., PEG-ceramides).

When used, suitable hydrophilic polymers for forming the conjugate include the hydrophilic polymers described above, preferably the polyalkyl ethers and more preferably polyoxyethylene glycol. In addition to tending to increase circulation half-life of the liposome, the hydrophilic polymer acts as a spacer which extends the antagonist away from the liposomal surface, thereby tending to increase binding of the liposome to the target site.

In addition to, or alternatively to the hydrophilic polymer, the conjugate may comprise other linking moieties chemically linking the lipid and antagonist, to act for example as spacers which tend to increase binding of the liposome to the target site. The linking moiety may directly or indirectly link the lipid and receptor antagonist. That is, a preferred conjugate construct can be described by the formula:

lipid-Xₐ-(polymer)_{b}-Y_{c}-antagonist

where lipid is a lipid material such as described above,

X is a linking moiety,

polymer is a hydrophilic polymer such as described above,

Y is a linking moiety which may be the same or different from X,

antagonist is a receptor antagonist such as described above,

and a, b, and c are independently 0 or 1, wherein preferably at least one of a,

b and c is 1.

Suitable linking moieties have functional groups capable of chemical bonding, preferably covalently bonding, with the components being linked via the moiety. Suitable linking moieties include nitro phenyl carbonate, succinimidyl succinate, orthopyridyl-disulfide, benzotriazole carbonate, and oxycarbonylimidazole. The conjugate is typically formed by covalent bonding of the component molecules (i.e., lipid, antagonist, optional hydrophilic polymer, and optional linking moieties) through the formation of amide, thioether, hydrazone or imino groups between acid, aldehyde, hydroxy, amino, thio or hydrazide groups on the components of the conjugate. Amide-linkages are preferred for biostability. The lipids, antagonists, and hydrophilic polymer can be derivatized according to methods known in the art, if desired to provide particular reactive groups and linkages.

Methods of chemically linking a hydrophilic polymer and a lipid, and activating the free end of the polymer for reaction with a selected ligand are known in the art and are useful in the present invention. In general, the hydrophilic polymer is derivatized at its terminal to contain reactive groups capable of coupling with reactive groups present in the ligand, for example, sulfhydryl, amine, aldehyde, or ketone groups. Examples of hydrophilic polymer terminal reactive groups include maleimide, N-hydroxysuccinimide (NHS), NHS-carbonate ester, hydrazide, hydrazine, iodoacetyl and dithiopyridine. Suitable such techniques and/or synthetic reaction schemes are described in U.S. Pat. Nos. 5,013,556; 5,631,018; 5,527,528; and 5,395,619; and in Allen, T. M. et al., Biochimica et Biophysica Acta 1237:99-108 (1995); Zalipsky, S., Bioconjugate Chem., 4(4):296-299 (1993); Zalipsky, S. et al., FEBS Lett. 353:71-74 (1994); Zalipsky, S. et al., Bioconjugate Chemistry, 705-708 (1995); Zalipsky, S. in STEALTH LIPOSOMES (D. Lasic and F. Martins, Eds.) Chapter 9, CRC Press, Boca Raton, FL (1995).

Where the lipid and receptor antagonist are directly conjugated, in one embodiment the antagonist comprises a free amino group which is reacted with a free hydroxyl group on the lipid according to methods known in the art, e.g., as described in Bailey, A. L., Monck, M. A., Cullis, P. R. pH-Induced Destabilization of Lipid Bilayers By A Lipopeptide Derived From Influenza Hemagglutinin. Biochimica et Biophysica Acta 1324(2):232-44, 1997.

In one particular embodiment the conjugate comprises a hydrophilic polymer having a proximal end and a distal end, the polymer being chemically linked at its proximal end to the polar head group of the vesicle-forming lipid conjugate and chemically linked at its distal end to the antagonist. In further particular embodiments of such conjugates, the hydrophilic polymer is selected from polyalkylethers and alkoxy-capped analogs thereof (especially polyoxyethylene glycol and methoxy- or ethoxy-capped analogs thereof), or poly(sialic acid) and analogs thereof.

Preferred conjugates comprise:

(1) PEGylated DSPE and a vitronectin receptor antagonist (VRA), whereinthe PEG group links the DSPE and the antagonist, or

(2) PEGylated gemini surfactant and a vitronectin receptor antagonist, wherein the PEG group links the gemini surfactant and the antagonist, preferably PEGylated DSPE and a vitronectin receptor antagonist.

Particularly preferred liposomes of the invention comprise:

HSPC (10-90 mol %)

Cholesterol (0-60 mol %, also about 30 to about 50 mol %)

PEG-DSPE (0-20 mol %, also 0 to about 5 mol %)

VRA conjugate (0.5-20 mol %);

DSPC (10-90 mol %)

Cholesterol (0-60 mol %, also about 30 to about 50 mol %)

PEG-DSPE (0-20 mol %, also 0 to about 5 mol %)

VRA conjugate (0.5-20 mol %);

POPC (10-90 mol %)

Cholesterol (0-60 mol %, also about 30 to about 50 mol %)

PEG-DSPE (0-20 mol %, also 0 to about 5 mol %)

VRA conjugate (0.5-20 mol %);

Sphingomyelin (10-90 mol %)

Cholesterol (0-60 mol %, also about 30 to about 50 mol %)

PEG-DSPE (0-20 mol %, also 0 to about 5 mol %)—

VRA conjugate (0.5-20 mol %);

POPC (80-99.5 mol %)

PEG-DSPE (0-20 mol %, also 0 to about 5 mol %)

VRA conjugate (0.5-20 mol %); or

EggPC (80-99.5 mol %)

PEG-DSPE (0-20 mol %, also 0 to about 5 mol %)

VRA conjugate (0.5-20 mol %)

Preparation of liposomes is well known in the art and such known methods may be used in the present invention. In general, liposome formation involves preparing a mixture of vesicle-forming lipids in powder form, dissolving the mixture in an organic solvent, freeze-drying the solution (lyophilizing), removing traces of solvent, reconstituting the mixture with buffer to form multilamellar vesicles, and optionally extruding the solution through a filter to form large or small unilamellar vesicles. The pH, temperature and total lipid ratio are selected according to principles well known in the art so as to form the lipid bilayers. Examples of methods of forming liposomes suitable for use in the invention include those described by L. D. Mayer et al., Vesicles of Variable Sizes Produced by a Rapid Extrusion Procedure, B.B.A. 858(1); 161-8, 1986; Szoka, F., Jr. et al., Ann. Rev. Biophys. Bioeng. 9:467 (1980); and U.S. Pat. Nos. 5,077,056; 5,013,556; 5,631,018 and 5,395,619.

For ease of manufacture, the receptor antagonist-lipid conjugate is preferably incorporated into the liposomes during their preparation, i.e., the conjugate is present during formation of the bilayer. In this embodiment, the conjugate is included in the mixture of powdered lipid materials used to prepare the liposomes such as described above. The resulting liposomes tend to have the receptor antagonist present on both the inner and the outer surface of the lipid bilayer.

The present invention also contemplates forming the conjugate in situ by incubating the antagonist with one or more vesicle-forming lipids during formation of the lipid bilayer of the liposome, under conditions sufficient to chemically link the antagonist and a vesicle-forming lipid. Alternatively, the conjugate can be incorporated into the liposomes after their formation, i.e., the conjugate is inserted in the bilayer after formation of the bilayer. In this embodiment the antagonist tends to be present only on the external surface of the lipid bilayer. In this embodiment, the conjugate is dissolved in a suitable solvent and the resulting solution is incubated with the liposomes under gentle mixing (e.g., stirring) for a time effective for the conjugate to assemble in the liposomes' lipid bilayer. In this embodiment, commercially available liposomes, including STEALTH® liposomes and the like, may be used. Alternatively the liposomes may be prepared by methods well known in the art. For example, a method of incorporating a targeting conjugate into a pre-formed liposome is set forth in U.S. Pat. No. 6,056,973 issued to Allen et al. on May 2, 2000.

The present invention also contemplates forming the conjugate in situ by incubating the antagonist with a pre-formed liposome comprising a vesicle-forming lipid under conditions sufficient to chemically link the antagonist and the vesicle-forming lipid.

In other aspects, the present invention also relates to conjugates and liposomes that are formed by the process of chemically linking, directly or indirectly, the required components and optionally the optional components described herein in regard to the conjugates and liposomes.

The liposomes preferably comprise a therapeutic or diagostic agent entrapped in the liposome for delivery to a disease site presenting the targeted receptor. Of course, selection of a particular agent will be made depending on the disease being treated or diagnosed. Selection of an active agent will be made based on the nature of the disease site and the activity of the agent toward that site, which may be based, for example, on chemosensitivity testing according to methods known in the art, or on historical information and accepted clinical practice.

Therapeutic agents may be selected, for example, from natural or synthetic compounds having the following activities: anti-angiogenic, anti-arthitic, anti-arrhythmic, anti-bacterial, anti-cholinergic, anti-coagulant, anti-diuretic, anti-epilectic, anti-fungal, anti-inflammatory, anti-metabolic, anti-migraine, anti-neoplastic, anti-parasitic, anti-pyretic, anti-seizure, anti-sera, anti-spansmodic, analgesic, anesthetic, beta-blocking, biological response modifying, bone metabolism regulating, cardiovascular, diuretic, enzymatic, fertility enhancing, growth-promoting, hemostatic, hormonal, hormonal suppressing, hypercalcemic alleviating, hypocalcemic alleviating, hypoglycemic alleviating, hyperglycemic alleviating, immunosuppressive, immunoenhancing, muscle relaxing, neurotransmitting, parasympathomimetic, sympathominetric plasma extending, plasma expanding, psychotropic, thrombolytic and vasodilating. Cytotoxic therapeutic agents are especially useful in the present invention.

Examples of therapeutic agents that can be delivered include topoisomerase I inhibitors, topoisomerase I/II inhibitors, anthracyclines, vinca alkaloids, platinum compounds, antimicrobial agents, quinazoline antifolates thymidylate synthase inhibitors, growth factor receptor inhibitors, methionine aminopeptidase-2 inhibitors, angiogenesis inhibitors, coagulants, cell surface lytic agents, therapeutic genes, plasmids comprising therapeutic genes, Cox II inhibitors, RNA-polymerase inhibitors, cyclooxygenase inhibitors, steroids, and NSAIDs (nonsteroidal anti-inflammatory agents).

Specific examples of therapeutic agents include:

Topoisomerase I-inhibiting camptothecins and their analogs or derivatives, such as SN-38 ((+)-(4S)4,11-diethyl4,9-dihydroxy-1H-pyrano[3′,4′:6,7]-indolizine[1,2-b]quinoline-3,14(4H,12H)-dione); 9-aminocamptothecin; topotecan (hycamtin; 9-dimethyl-aminomethyl-10-hydroxycamptothecin); irinotecan (CPT-11; 7-ethyl-10-[4-(1-piperidino)-1-piperidino]-carbonyloxy-camptothecin), which is hydrolyzed in vivo to SN-38); 7-ethylcamptothecin and its derivatives (Sawada, S. et al., Chem. Pharm. Bull., 41(2):310-313 (1993)); 7-chloromethyl-10,11-methylene-dioxy-camptothecin; and others (SN-22, Kunimoto, T. et al., J. Pharmacobiodyn., 10(3): 148-151 (1987); N-formylamino-12,13,dihydro-1,11-dihydroxy-13-(beta-D-glucopyransyl)-5H-indolo[2,3-a]pyrrolo[3,4-c]carbazole-5,7(6H)-dione (NB-506, Kanzawa, G. et al., Cancer Res., 55(13):2806-2813 (1995); DX-8951 f and lurtotecan (GG-211 or 7-(4-methylpiperazino-methylene)-10,11-ethylenedioxy-20(S)-camptothecin) (Rothenberg, M. L., Ann. Oncol., 8(9):837-855 (1997)); 7-(2-(N-isopropylamino)ethyl)-(20S)-camptothecin (CKD602, Chong Kun Dang Corporation, Seoul Korea);

Topoisomerase I/II-inhibiting compounds such as 6-[[2-dimethylamino)-ethyl]amino]-3-hydroxy-7H-indeno[2,1-c]quinolin-7-one dihydrochloride, (TAS-103, Utsugi, T., et al., Jpn. J. Cancer Res., 88(10):992-1002 (1997)); 3-methoxy-11H-pyrido[3′,4′-4,5]pyrrolo[3,2-c]quinoline-1,4-dione (AzalQD, Riou, J. F., et al., Mol. Pharmacol., 40(5):699-706 (1991));

Anthracyclines such as doxorubicin, daunorubicin, epirubicin, pirarubicin, and idarubicin;

Vinca alkaloids such as vinblastine, vincristine, vinleurosine, vinrodisine, vinorelbine, and vindesine;

Platinum compounds such as cisplatin, carboplatin, ormaplatin, oxaliplatin, zeniplatin, enloplatin, lobaplatin, spiroplatin, ((-)-(R)-2-aminomethylpyrrolidine (1,1-cyclobutane dicarboxylato)platinum), (SP-4-3(R)-1,1-cyclobutane-dicarboxylato(2-)-(2-methyl-1,4-butanediamine-N,N′)platinum), nedaplatin, and (bis-acetato-ammine-dichloro-cyclohexylamine-platinum(IV));

Anti-microbial agents such as gentamicin and nystatin;

Quinazoline antifolates thymidylate synthase inhibitors such as described by Hennequin et al. Quinazoline Antifolates Thymidylate Synthase Inhibitors: Lipophilic Analogues with Modification to the C2-Methyl Substituent (1996) J. Med. Chem. 39, 695-704;

Growth factor receptor inhibitors such as described by: Sun L. et al., Identification of Substituted 3-[(4,5,6,7-Tetrahydro-1H-indol-2-yl)methylene]-1,3-dihydroindol-2-ones as Growth Factor Receptor Inhibitors for VEGF-R2 (Flk-1/KDR), FGF-R1, and PDGF-Rbeta Tyrosine Kinases (2000) J. Med. Chem. 43:2655-2663; and Bridges A. J. et al. Tyrosine Kinase Inhibitors. 8. An Unusually Steep Structure-Activity Relationship for Analogues of 4-(3-Bromoanilino)-6,7-dimethoxyquinazoline (PD 153035), a Potent Inhibitor of the Epidermal Growth Factor Receptor (1996) J. Med. Chem. 39:267-276,

Inhibitors of angiogenesis, such as angiostatin, endostatin, echistatin, thrombospondin, plasmids containing genes which express anti-angiogenic proteins, and methionine aminopeptidase-2 inhibitors such as fumagillin, TNP-140 and derivatives thereof;

and other therapeutic compounds such as 5-fluorouracil (5-FU), mitoxanthrone, cyclophosphamide, mitomycin, streptozocin, mechlorethamine hydrochloride, melphalan, cyclophosphamide, triethylenethiophosphoramide, carmustine, lomustine, semustine, hydroxyurea, thioguanine, decarbazine, procarbazine, mitoxantrone, steroids, cytosine arabinoside, methotrexate, aminopterin, motomycin C, demecolcine, etopside, mithramycin, Russell's Viper Venom, activated Factor IX, activated Factor X, thrombin, phospholipase C, cobra venom factor [CVF], and cyclophosphamide.

Preferred therapeutic agents are selected from: antineoplastic agents, such as topotecan, doxorubicin, daunorubicin, vincristine, mitoxantrone, carboplatin, RNA-polymerase inhibitors, and combinations thereof; anti-inflammatory agents, such as cyclooxygenase inhibitors, steroids, and NSAIDs; anti-angiogenesis agents such as fumagillin, tnp-140, cyclooxygenase inhibitors, angiostatin; endostatin, and echistatin; anti-infectives; and combinations thereof. In a particular embodiment, the therapeutic active is selected from the group consisting of topotecan, doxorubicin, daunorubicin, vincristine, mitoxantrone, RNA-polymerase inhibitors, and combinations thereof, especially topotecan. Other camptothecins, and camptothecin analogs, are also especially useful therapeutic actives.

Examples of diagnostic agents include contrast agents for imaging including paramagnetic, radioactive or fluorogenic ions. Specific examples of such diagnostic agents include those disclosed in U.S. Pat. No. 5,855,866 issued to Thorpe et al. on Jan. 5, 1999.

Methods of incorporating therapeutic and diagnostic agents into liposomes are well known in the art and are useful in the present invention. Suitable methods include passive entrapment by hydrating a lipid film with an aqueous solution of a water-soluble agent or by hydrating a lipid film containing a lipophilic agent, pH/ion gradient loading/retention (e.g., ammonium sulfate gradients), polymer gradient loading/retention, and reverse phase evaporation liposome preparation. For example, useful methods of loading such agents are described in Haran, G. et al., Transmembrane Ammonium Sulfate Gradients in Liposomes Produce Efficient and Stable Entrapment of Amphipathic Weak Bases, Biochim Biophys Acta, Vol 151, pp 201-215 (1993); U.S. Pat. No. 5,077,056 issued to Bally et al. on Dec. 31, 1991; PCT Publication No. WO 98/17256, published Apr. 30, 1998; Zhu, et al., The Effect of Vincristine-Polyanion Complexes 1N STEALTH Liposomes on Pharmacokinetics, Toxicity and Anti-Tumor Activity, Cancer Chemother Pharmacol (1996) 39:138-142; and PCT Publication No. WO 00/23052. The agents can be incorporated into one or more of the liposomal compartments, or be bound to the liposome membrane.

In order to use the liposomes of the invention, they will normally be formulated into a pharmaceutical composition, in accordance with standard pharmaceutical practice. This invention therefore also relates to a pharmaceutical composition, comprising (a) an effective, non-toxic amount of the liposomes herein described and (b) a pharmaceutically acceptable carrier or diluent.

The liposomes of the invention and pharmaceutical compositions incorporating such may conveniently be administered by any of the routes conventionally used for drug administration, for instance, parenteral, oral, topical, by inhalation (e.g., intertracheal), subcutaneous, intramuscular, interlesional (e.g., to tumors), intemasal, intraocular, and by direct injection into organs and intravenous. Parenteral, particularly intravenous administration is preferred. Where the liposomes are designed to provide anti-angiogenic activity, administration will preferably be by a route involving circulation of the liposomes in the bloodstream, including intravenous administration.

The liposomes may be administered in conventional dosage forms prepared by combining the liposomes with standard pharmaceutical carriers according to conventional procedures. The liposomes may also be administered in conventional dosages in combination with one or more other therapeutically active or diagnostic compounds. These procedures may involve mixing, granulating and compressing or dissolving the ingredients as appropriate to the desired preparation.

It will be appreciated that the form and character of the pharmaceutically acceptable carrier or diluent is dictated by the amount of liposome and other active agents with which it is to be combined, the route of administration and other well-known variables. The carrier(s) must be “acceptable” in the sense of being compatible with the other ingredients of the formulation and not deleterious to the recipient thereof. The liposomes will typically be provided in suspension form in a liquid carrier such as aqueous saline or buffer. In general, the pharmaceutical form will comprise the liposomes in an amount sufficient to deliver the liposome or loaded compound in the desired dosage amount and regimen.

The liposomes are administered in an amount sufficient to deliver the liposome or loaded compound in the desired dosage according to the desired regimen, to ameliorate or prevent the disease state which is being treated, or to image the disease site being diagnosed or monitored.

It will be recognized by one of skill in the art that the optimal quantity and spacing of individual dosages of the liposomes will be determined by the nature and extent of the condition being treated, diagnosed or monitored, the form, route and site of administration, and the particular patient being treated, and that such optimums can be determined by conventional techniques. It will also be appreciated by one of skill in the art that the optimal course of treatment, i.e., the number of doses of the liposomes given per day for a defined number of days, can be ascertained by those skilled in the art using conventional course of treatment determination tests.

Once administered, the liposomes associate with the targeted tissue, or are carried by the circulatory system to the targeted tissue, where they associate with the tissue. At the targeted tissue site, the receptor antagonist may itself exhibit clinical efficacy, that is, the liposomes per se may be useful in treating disease presenting the targeted receptors. As will be appreciated by those skilled in the art, the selection of the liposome is based on the expression of the conjugate's cognate receptor on a patient's diseased cells, which can be determined by known methods or which may be based on historical information for the disease.

In addition or alternatively, the therapeutic or diagnostic agent associated with the liposomes is released or diffuses to the targeted tissue where it performs its intended function.

For example, liposomes comprising a receptor antagonist to receptors upregulated in the vascular endothelium of disease sites, such as inflammation, infection or tumor sites (e.g., the vitronectin receptor), are useful for treating diseases characterized by neovascularization (angiogenesis). Such diseases include osteo and rheumatoid arthritis, diabetic retinopathy, hemangiomas, psoriasis, restenosis and cancerous tumors (solid primary tumors as well as metastatic disease). The receptor antagonist binds the vitronectin receptor present at the disease site to inhibit formation of vasculature, which supports the disease state or symptoms. For treating or diagnosing such diseases, the liposomes will preferably comprise a therapeutic agent and/or diagnostic agent selected from the group consisting of anti-inflammatory agents, anti-neoplastic agents, anti-infectives, anti-angiogenic agents, and/or a diagnostic imaging agent. Selection of an active agent will be made based on the nature of the disease site (e.g., tumor, inflammation or infection) and the activity of the agent toward that site (e.g., anti-neoplastic, anti-inflammatory, anti-infective, respectively). Selection of a particular agent may be based on chemosensitivity testing according to methods known in the art, or may be based on historical information and accepted clinical practice. For example, topotecan is known to be an active agent against ovarian cancer, and therefore is useful for treatment of ovarian cancer based on accepted clinical practice.

### EXAMPLES

The following abbreviations are used in the experimental section:

VRA—vitronectin receptor antagonist

DSPE—distearylphosphatidylethanolamine

PEG—polyethylene glycol

### Example 1

Preparation of the VRA (S)-7-[[N-(4-Aminobutyl)-N-(benzimidazol-2-ylmethyl)] amino]carbonyl-4-methyl-3-oxo-2,3,4,5-tetrahydro-1H-1,4-benzodiazepine-2-acetic acid:

General

Proton nuclear magnetic resonance (¹H NMR) spectra are recorded at either 300 or 400 MHz, and chemical shifts are reported in parts per million (δ) downfield from the internal standard tetramethylsilane (TMS). Mass spectra are obtained using electrospray (ES) ionization techniques. Elemental analyses are performed by Quantitative Technologies Inc., Whitehouse, N.J. All temperatures are reported in degrees Celsius. Analtech Silica Gel GF and E. Merck Silica Gel 60 F-254 thin layer plates are used for thin layer chromatography. Flash chromatography is carried out on E. Merck Kieselgel 60 (230400 mesh) silica gel. Analytical and preparative HPLC is performed on Beckman chromatography systems. ODS refers to an octadecylsilyl derivatized silica gel chromatographic support. YMC ODS-AQ® is an ODS chromatographic support and is a registered trademark of YMC Co. Ltd., Kyoto, Japan. PRP-1 ® is a polymeric (styrene-divinylbenzene) chromatographic support, and is a registered trademark of Hamilton Co., Reno, Nev. Celite® is a filter aid composed of acid-washed diatomaceous silica, and is a registered trademark of Manville Corp., Denver, Colo.

The title VRA is synthesized in accordance with the following scheme 1:

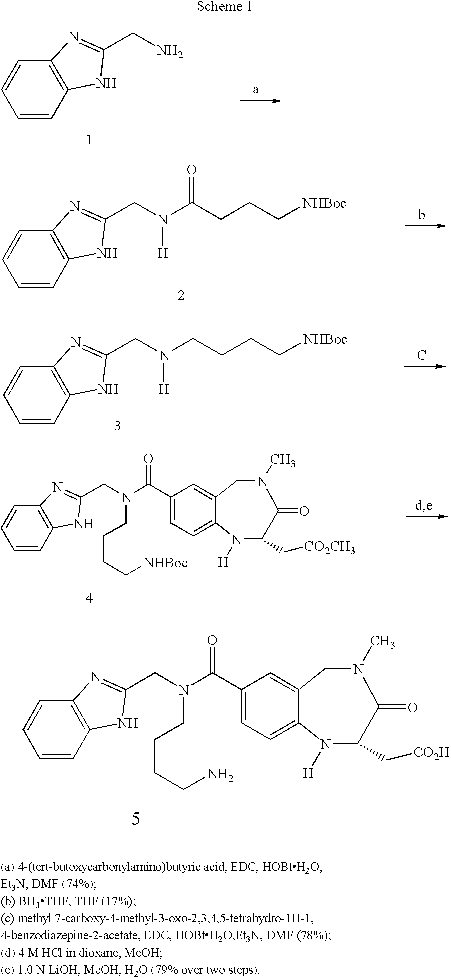

a) N-(Benzimidazol-2-Ylmethyl)4-(Tert-Butoxycarbonylamino)Butyramide

4-(tert-Butoxycarbonylamino)butyric acid (5.0 g, 24.6 mmole), 2-aminomethylbenzimidazole dihydrochloride hydrate (6.5 g, 29.5 mmole), EDC (5.7 g, 29.5 mmole), HOBt.H₂O (3.99 g, 29.5 mmole), and Et₃N (17 mL, 123 mmole) are combined in DMF (120 mL) at RT. The reaction is stirred for 18 hr, then is concentrated to dryness. The residue is purified by flash chromatography on silica gel to afford the title compound (6.04 g, 74%): ¹H NMR (400 MHz, CDCl₃) δ 7.40-7.80 (m, 2 H), 7.29-7.38 (m, 1 H), 7.20-7.27 (m, 2 H), 4.77-4.90 (m, 1 H), 4.69 (d, J=5.8Hz, 2 H), 3.11-3.22 (m, 2 H), 2.20-2.39 (m, 2 H), 1.77-1.88 (m, 2 H), 1.44 (s, 9 H).

b) N-(Benzimidazol-2-Ylmethyl)-N-[4-(Tert-Butoxycarbonylamino)Butyl]Amine

Borane-tetrahydrofuran complex (1.0 M in THF, 55 mL, 55 mmole) is added slowly to a suspension of N-(benzimidazol-2-ylmethyl)-4-(tert-butoxycarbonylamino)butyramide (6.04 g, 18.2 mmole) in THF (90 mL) at RT. The resulting homogeneous solution is heated at reflux for 18 hr, then cooled to RT. A solution of 5% AcOH in EtOH is added, and the solution is stirred for 18 hr. The resulting solution is concentrated to dryness and the residue is taken up in saturated NaHCO₃. The mixture is extracted with CH₂Cl₂ (4×), and the combined organic layers are dried (MgSO₄) and concentrated. Flash chromatography on silica gel (10% MeOH/CH₂Cl₂) gives the title compound (985 mg, 17%) as a light tan gum:

¹H NMR (400 MHz, CDCl₃) δ 7.53-7.63 (m, 2 H), 7.18-7.30 (m, 2 H), 4.12 (s, 2 H), 3.00-3.18 (m, 2 H), 2.65-2.75 (m, 2 H), 1.35-1.63 (m, 13 H).

c) Methyl (S)-7-[[N-(Benzimidazol-2-Ylmethyl)-N-[4-(Tert-Butoxycarbonylamino)Butyl]Amino]Carbonyl-4-Methyl-3-Oxo-2,3,4,5-Tetrahydro-1H-1,4-Benzodiazepine-2-Acetate

Methyl 7-carboxy-4-methyl-3-oxo-2,3,4,5-tetrahydro-1H-1,4-benzodiazepine-2-acetate is synthesized by the method described in William H Miller, et al.,: Enantiospecific Synthesis of SB 214857, a Potent, Orally Active, Nonpeptide Fibrinogen Receptor Antagonist Tetrahedron Letters (1995) 36(52): 9433-9436.

Methyl 7-carboxy4-methyl-3-oxo-2,3,4,5-tetrahydro-1H-1,4-benzodiazepine-2-acetate (753 mg, 2.6 mmole), N-(benzimidazol-2-ylmethyl)-N-[4-(tert-butoxycarbonylamino)butyl]amine (985 mg, 3.1 mmole), EDC (594 mg, 3.1 mmole), HOBt.H₂O (419 mg, 3.1 mmole), and Et₃N (0.90 mL, 6.5 mmole) are combined in DMF (15 mL) at RT. The reaction is stirred for 18 hr, then is concentrated to dryness. The residue is purified by flash chromatography on silica gel (5% MeOH/CH₂Cl₂) to afford the title compound (1.2 g, 78%) as a light tan solid: ¹H NMR (400 MHz, CDCl₃) δ 10.55 (br s, 1 H), 7.75 (d, J=8.5 Hz, 1 H), 7.45 (d, J=8.5 Hz, 1 H), 7.20-7.32 (m, 2 H), 7.10-7.20 (m, 2 H), 6.52 (d, J=8.1 Hz, 1 H), 5.43 (d, J=16.5 Hz, 1 H), 5.02-5.12 (m, 1 H), 4.73-4.85 (m, 2 H), 4.55-4.65 (m, 1H), 4.49 (d, J=4.7 Hz, 1 H), 3.74 (s, 3 H), 3.70 (d, J=16.5 Hz, 1 H), 3.36-3.46 (m, 2 H), 3.04 (s, 3 H), 2.90-3.10 (m, 3 H), 2.67 (dd, J=16.0, 6.4 Hz, 1 H), 1.60-1.75 (m, 2 H), 1.43 (s, 9 H), 1.17-1.32 (m, 2 H); MS (ES) m/e 593 (M+H)⁺.

d) (S)-7-[[N-(4-Aminobutyl)-N-(Benzimidazol-2-Ylmethyl)]Amino]Carbonyl-4-Methyl-3-Oxo-2,3,4,5-Tetrahydro-1H-1,4-Benzodiazepine-2-Acetic Acid

4 M HCl in dioxane (30 mL, 120 mmole) is added to a solution of methyl (S)-7-[[N-(benzimidazol-2-ylmethyl)-N-[4-(tert-butoxycarbonylamino)butyl]amino]carbonyl-4-methyl-3-oxo-2,3,4,5-tetrahydro-1H-1,4-benzodiazepine-2-acetate (1.2 g, 2 mmole) in MeOH (10 mL) at RT. After 2 hr, the solution is concentrated to dryness to leave an off-white powder (1.24 g). This powder is dissolved in MeOH/H₂O (10 mL), and 1.0 N LiOH (10 mL, 10 mmole) is added. The reaction is stirred at RT for 18 hr, then concentrated to dryness. The residue is taken up in H₂O and the pH is adjusted to about 5 with 10% HCl. The precipitated solid is collected by suction filtration and washed with H₂O. Drying in high vacuum gives the title compound (760 mg, 79%) as a white solid: ¹H NMR (400 MHz, CDCl₃) δ 7.48-7.68 (m, 2 H), 7.05-7.35 (m, 4 H), 6.57 (d, J=8.2 Hz, 1 H), 5.51 (d, J=16.0Hz, 1H), 5.12 (t, J=6.8 Hz, 1 H), 4.70-5.00 (m, 2 H, obscured by residual solvent signal), 3.62-3.90 (m, 1 H), 3.40-3.62 (m, 2 H), 2.95 (s, 3 H), 2.69-3.00 (m, 3 H), 2.45 (dd, J=15.6, 6.6 Hz, 1 H), 1.60-1.80 (m, 2 H), 1.30-1.60 (m, 2 H); MS (ES) m/e 479 (M+H)⁺. Anal. Calcd for C₂₅H₃₀N₆O₄.²H₂O: C, 58.35; H, 6.63; N, 16.33. Found: C, 58.17; H, 6.63; N, 16.11.

Analogous VRAs having a functional aliphatic carboxylic acid group or aliphatic sulfhydryl group instead of the aliphatic amino group can be prepared in a similar manner, substituting the appropriate carboxylic acid in step (a) and utilizing the solvents 4M HCl in dioxane, CH₂Cl₂ in step (d).

The title VRA is alternatively synthesized in accordance with the following scheme 2:

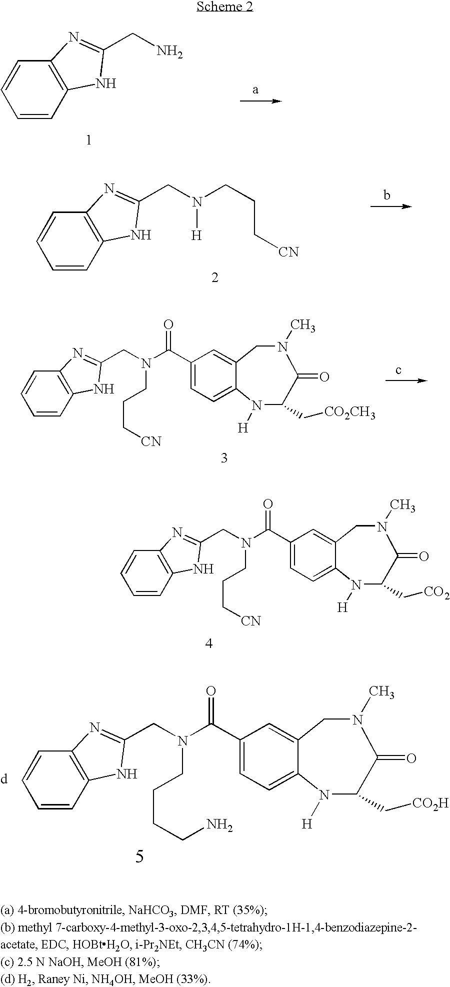

a) 4-[(Benzimidazol-2-Ylmethyl)Amino]Butyronitrile

To a stirred mixture of 2-aminomethylbenzimidazole dihydrochloride hydrate (0.5 g, 2.2717 mmole) and NaHCO₃ (0.67 g, 7.951 mmole) in dry DMF (10 mL) is added 4-bromobutyronitrile (0.37 g, 2.4989 mmole). After stirring at RT for 24 hr, the mixture is concentrated. The residue is taken up in H₂O and extracted with CH₂Cl₂. The organic extracts are dried over MgSO4, concentrated, and purified by silica gel flash column chromatography (5% MeOH/CH₂Cl₂) to give the title compound (0.15 g, 35%) as abrown oil: ¹H NMR (250 MHz, DMSO-d₆) δ 7.50(m, 2H),7.14(m, 2H),4.11 (s, 2H),2.85 (t, J=4Hz, 2H),2.45 (t, J=4Hz, 2H),1.82(m, 2H).

b) Methyl (S)-7-[[N-(Benzimidazol-2-Ylmethyl)-N-(3-Cyanopropyl)]Amino]Carbonyl4-Methyl-3-Oxo-2,3,4,5-Tetrahydro-1H-1,4-Benzodiazepine-2-Acetate

To a stirred mixture of 4-[(benzimidazol-2-ylmethyl)amino]butyronitrile (0.159 g, 0.7422 mmole), methyl 7-carboxy-4-methyl-3-oxo-2,3,4,5-tetrahydro-1H-1,4-benzodiazepine-2-acetate (0.217 g, 0.7422 mmole), HOBt.H₂O (0.120 g, 0.8906 mmole), and i-Pr₂NEt (0.192 g, 1.4844 mmole) in dry CH₃CN (7 mL) is added EDC (0.265 g, 0.8906 mmole). After stirring at RT for 48 hr, the mixture is concentrated. The residue is taken up in H₂O and extracted with CH₂Cl₂. The organic layer is washed sequentially with saturated NaHCO₃ and brine, dried over MgSO₄, and concentrated to give a brown oil. Silica gel flash column chromatography (3% MeOH/CH₂Cl₂) gives the title compound (0.261 g, 74%) as an off white foam: ¹H NMR (250 MHz, DMSO-d₆): δ 7.62 (m, 1 H), 7.50 (m, 1 H), 7.25 (m, 4 H), 6.54 (d, J=8.3 Hz, 1H), 6.40 (d, J=3.5 Hz, 1H), 5.48 (d, J=16 Hz, 1 H), 5.15 (m, 1 H), 4.84 (d, J=2.9 Hz, 2 H), 4.52 (s, 2 H), 3.80 (d, J=16 Hz, 1 H), 3.60 (s, 3 H), 3.45 (t, J=8.7 Hz, 2 H), 2.85 (t, J=8.7 Hz, 2 H), 2.78 (dd, J=16.4, 3.5 Hz, 1 H), 2.66 (dd, J=16.4, 3.5 Hz, 1 H), 1.95 (m, 2 H).

c) (S)-7-[[N-(Benzimidazol-2-Ylmethyl)-N-(3-Cyanopropyl)]Amino]Carbonyl-4-Methyl-3-Oxo-2,3,4,5-Tetrahydro-1H-1,4-Benzodiazepine-2-Acetic Acid

To a stirred solution of methyl (S)-7-[[N-(benzimidazol-2-ylmethyl)-N-(3-cyanopropyl)]amino]carbonyl-4-methyl-3-oxo-2,3,4,5-tetrahydro-1H-1,4-benzodiazepine-2-acetate (0.261 g, 0.5478 mmole) in MeOH (5 mL) is added 2.5 N NaOH (0.7 mL, 1.6433 mmole). After stirring at RT overnight, the mixture is concentrated. The residue is taken up in H₂O, and the solution is acidified with 6 N HCl to pH=4. The white solid is filtered and dried to afford the title compound (0.21 g, 81%): ¹H NMR (250 MHz, DMSO-d₆): δ 7.62 (m, 1 H), 7.50 (m, 1 H), 7.25 (m, 4 H), 6.54 (d, J=8.3 Hz, 1 H), 6.40 (d, J=3.5 Hz, 1 H), 5.48 (d, J=16 Hz, 1 H), 5.15 (m, 1 H), 4.84 (d, J=2.9 Hz, 2 H), 4.52 (s, 2 H), 3.80 (d, J=16Hz, 1 H), 3.45 (t, J=8.7 Hz, 2 H), 2.85 (t, J=8.7 Hz, 2 H), 2.78 (dd, J=16.4, 3.5 Hz, 1 H), 2.66 (dd, J=16.4, 3.5 Hz, 1H), 1.95 (m, 2 H).

d) (S)-7-[[N-(4-Aminobutyl)-N-(Benzimidazol-2-Ylmethyl)]Amino]Carbonyl-4-Methyl-3-Oxo-2,3,4,5-Tetrahydro-1H-1,4-Benzodiazepine-2-Acetic Acid

A mixture of (S)-7-[[N-(benzimidazol-2-ylmethyl)-N-(3-cyanopropyl)]amino]carbonyl-4-methyl-3-oxo-2,3,4,5-tetrahydro-1H-1,4-benzodiazepine-2-acetic acid (0.200 g, 0.4325 mmole) and NH₄OH (1 mL, 30% solution) in MeOH (5 mL) is hydrogenated over Raney Ni at RT for 24 hr. The catalyst is filtered off, and the filtrate is concentrated and purified by reverse phase chromatography (10% CH₃CN/H₂O containing 0.1% TFA) to give the title compound (0.100 g, 33%) as an off white solid: ¹H NMR (400 MHz, DMSO-d₆) δ 7.85 (m, 2 H), 7.75 (s, 2 H), 7.61 (m, 2 H), 7.20 (m, 2 H), 6.65 (d, J=8.3 Hz, 1H), 5.48 (d, J=16 Hz, 1 H), 5.15 (m, 1 H), 5.05 (s, 2 H), 3.85 (d, J=16 Hz, 1 H), 3.65 (t, J=8.7 Hz, 2 H), 2.95 (s, 3 H), 2.75 (dd, J=16.4, 3.5 Hz, 1 H), 2.70 (m, 2 H), 2.54 (dd, J=16.4, 3.5 Hz, 1 H), 1.72 (m, 2 H), 1.45 (m, 2 H); IR (KBr) 3425, 3000, 3100, 1728, 1675, 1630, 1625, 1613 cm⁻¹; MS (ES) m/e 479 (M+H)⁺. Anal. Calcd for C₂₅H₃₀N₆O₄.2CF₃CO₂H: C, 49.30; H, 4.56; N,11.89. Found: C, 49.22; H, 4.89; N, 11.84.

VRAs having a functional aliphatic carboxylic acid group or aliphatic sulfhydryl group are prepared in a similar manner using standard synthetic chemistry techniques, for example, according to the following schemes:

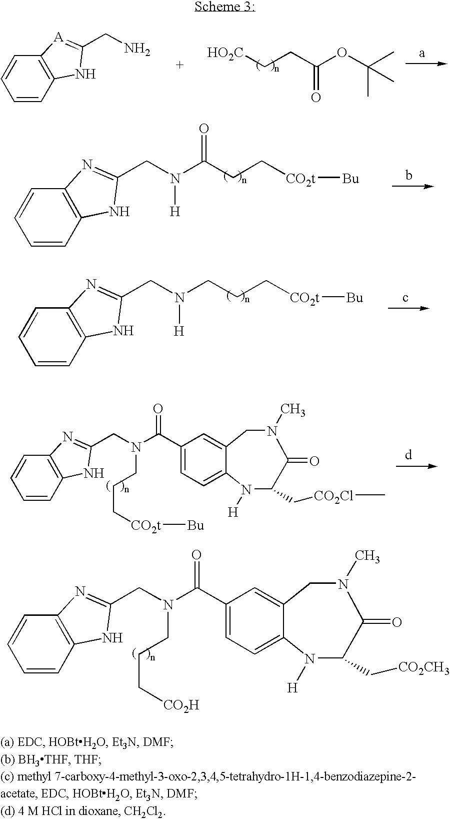

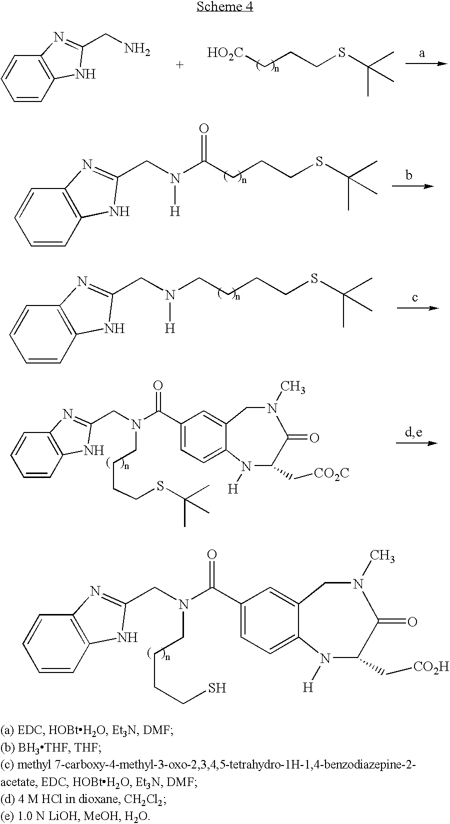

A VRA according to scheme 3 is coupled to a liposome-forming lipid or liposome via the VRA free carboxylic acid group, e.g., in the presence of 1.0 N LiOH, MeOH, H₂O. A VRA according to scheme 4 is coupled to a liposome-forming lipid or liposome via the VRA free sulfhydryl group.

### Example 2

Preparation of VRA-Lipid Conjugate:

Synthesis of a vitronectin receptor antagonist-lipid conjugate comprising the VRA of Example 1 is illustrated in FIG. 1.

[image]

DSPE-PEG-VRA is synthesized by reacting 50 mg of the VRA (2) with DSPE-PEG-NHS (1) (commercially available from Shearwater Polymers, Huntsville, Ala.) in 10 mL DMSO. Excess amount of VRA (1.2 times molar excess) is used. The VRA is completely dissolved in DMSO. DSPE-PEG-NHS pre-dissolved in DMSO is added dropwise to the VRA solution. This reaction mixture is stirred overnight in the dark at room temperature. The unreacted DSPE-PEG-NHS is quenched by the addition of excess glycine (5 times molar excess). The reaction mixture is diluted with 40 mL 0.1 M MES (morpholino ethanesulfonic acid) saline buffer (pH 5.8) and then dialyzed against the MES buffer (pH 5.8) to remove by-product, DMSO, and unreacted VRA (t this point the unreacted DSPE-PEG-NHS will be hydrolyzed into DSPE-PEG-COOH). The reaction mixture is then dialyzed against water and then lyophilized. The formation of DSPE-PEG-VRA is confirmed by matrix-assisted-laser-desorption/ionization (MALDI) mass spectrometry: estimated MW (Da)=4625; determined MW (Da)=4380. DSPE-PEG-COOH is removed from the DSPE-PEG-VRA using either ion exchange or reverse phase chromatography. The ratio of VRA to DSPE in this conjugate should be 1.

### Example 3

Preparation of VRA-Targeted Liposomes:

Liposomes comprising the lipid-VRA conjugate of Example 2 are prepared as follows. The composition of the lipid materials is shown in Table 1.

TABLE 1

| lipid material | mol % | | | | |
|---|---|---|---|---|---|
| | 3a | 3b | 3c | 3d | 3e |
| VRA - lipid conjugate | 0.5 | 1 | 2 | 5 | 10 |
| of Ex. 2 | | | | | |
| DSPC | 54.5 | 54 | 53 | 50 | 40 |
| cholesterol | 45 | 45 | 45 | 45 | 45 |
| | 3f | 3g | 3h | 3i | 3j |
| VRA - lipid conjugate | 0.5 | 1 | 2 | 5 | 10 |
| of Ex. 2 | | | | | |
| DPPC | 54.5 | 54 | 53 | 50 | 40 |
| cholesterol | 45 | 45 | 45 | 45 | 45 |
| | 3k | 3l | 3m | 3n | 3o |
| VRA - lipid conjugate | 0.5 | 1 | 2 | 5 | 10 |
| of Ex. 2 | | | | | |
| POPC | 54.5 | 54 | 53 | 50 | 40 |
| cholesterol | 45 | 45 | 45 | 45 | 45 |

The lipid materials are individually weighed and combined into an appropriately sized vessel. The lipids are completely dissolved in organic solvent, e.g. CHCl₃/MeOH 95/5 v/v, Benzene:MeOH 70/30 v/v, or ethanol. The solvent is evaporated off (or lyophilized in the case of benzene methanol) and trace solvent is removed under high vacuum. The lipid film is resuspended in aqueous buffer containing 20 mM Hepes, 150 mM NaCl pH 7.4 (HBS) at 65 degrees celcius with vortexing. The lipid suspension is sized by extrusion through 2-100 nm diameter polycarbonate filters to form ˜100 nm diameter vesicles.

Additional liposomes are prepared from the components shown in Table 2, which reflects the target mol % composition and the target weights of each component employed:

TABLE 2

approximate mol %

| | | | | 3s |
|---|---|---|---|---|
| component | 3p | 3q | 3r | (control) |
| VRA - lipid conjugate | 7 | 5 | 3 | 0 |
| of Ex. 2 | | | | |
| POPC | 53 | 53 | 53 | 53 |
| cholesterol | 40 | 40 | 40 | 40 |
| Pegylated DSPE* | 0 | 2 | 4 | 7 |

wt (mg)

| | | | | 3s |
|---|---|---|---|---|
| component | 3p | 3q | 3r | (control) |
| VRA - lipid conjugate | 7.42 | 5.36 | 3.25 | 0 |
| of Ex. 2 | | | | |
| POPC | 9.09 | 9.19 | 9.30 | 9.46 |
| cholesterol | 3.49 | 3.53 | 3.57 | 3.63 |
| Pegylated DSPE* | 0 | 1.92 | 3.88 | 6.91 |

*PEG3400 DSPE, commercially available from Shearwater Polymers, Huntsville, AL as DSPE-PEG-NHS, MW 3400.

The lipid materials are individually weighed and combined into an appropriately sized vessel. The lipids are completely dissolved in organic solvent, e.g. CHCl₃/MeOH 95/5 v/v, Benzene:MeOH 70/30 v/v, or ethanol. The solvent is evaporated off (or lyophilized in the case of benzene methanol) and trace solvent is removed under high vacuum. The lipid film is resuspended in TRIS buffered saline, (TBS: 50 mM TRIS, 100 mM NaCl pH 7.4) at 65 degrees celcius with vortexing. The lipid suspension is sized by extrusion through 2-100 nm diameter polycarbonate filters to form ˜100 nm diameter vesicles.

The liposomes are physically characterized for size and lipid composition using techniques known in the art:

a) Size by Dynamic Light Scattering

Samples are diluted to 1 mM with HBS and standard dynamic light scattering (zeta-sizing) is performed using a Malvern Zeta-Sizer.

b) Final lipid composition by HPLC

Final lipid composition is determined by HPLC methods using a normal phase Zorbax-SIL column. Lipid species are separated on a Hexane:Isopropanol:Water-Hexane:Isopropanol gradient; peak areas are quantitated by comparison with standards run on the same gradient and used to determine the final lipid composition.

### Example 4

In Vitro Binding Affinity of Liposomes of the Invention:

Liposomes of example 3 are tested for their binding affinity to human αVβ3 or αVβ5 using an in vitro solid phase binding assay previously described [Wong A, Hwang S M, McDevitt P, McNulty D, Stadel J M and Johanson K, Studies on alphavbeta 3/ligand interactions using a (³H)SK&F-107260 binding assay (1996) Molecular Pharmacology 50(3):529-537].

In vitro binding affinity of the liposomes to other receptors, or of liposomes comprising other ligands to receptors may be determined by receptor binding assays such as are known in the art.

Liposomes of the present invention are those having a Ki according to the receptor binding assay in the nanomolar to micromolar range, preferably in the nanomolar range.

Liposomes prepared according to Example 3, compositions 3p-3s, exhibited the following Ki values according to the above referenced binding assay published by Wong et al.[(1996) Molecular Pharmacology 50(3):529-537]:

| Example | Ki (nm) |
|---|---|
| 3p | 31 |
| 3q | 50 |
| 3r | 50 |
| 3s (control) | no binding effect |

In Vivo Biodistribution of Liposomes in Normal and Tumor-Bearing Animals:

Liposomes are prepared as in Example 3 with the following exceptions. Trace quantities of ³H-labelled cholesterylhexadecylether (CHE) are included in the liposomal membrane and used as a liposomal tracer for in vivo experiments; liposomes are sterile filtered prior to in vivo administration.

Liposomal biodistribution is tested in female C57Bl/6 normal or tumor bearing mice. Mice are given a bolus, intravenous injection of a buffered suspension of the liposomes via the lateral tail veil at a dose of ˜100 mg/kg body weight. Animals are sacrificed and blood and tissues are removed according to a defined timepoint schedule: 1, 4, 8, 12 and 24 hours following liposome administration. More specifically, blood is removed via cardiac puncture and placed in an EDTA-coated microtainer tube. Tubes are well mixed and plasma is separated from whole blood by centrifugation. Lung, liver, spleen, heart and kidneys are excised, and plasma and tissues are analyzed for the presence of radioactivity according to Monck M A. Mori A. Lee D. Tam P. Wheeler J J. Cullis P R. Scherrer P. (2000) Stabilized plasmid-lipid particles: pharmacokinetics and plasmid delivery to distal tumors following intravenous injection. Journal of Drug Targeting. 7(6):439-52, 2000. The tumor tissue should exhibit an accumulation of the labeled liposomes.

### Example 6

Treatment of Ovarian Cancer Using Liposomes of the Present Invention:

Liposomes as prepared in Example 3 are loaded with topotecan using ion gradient or polymer gradient loading/retaining techniques such as are known in the art. An aqueous saline suspension of the liposomes is administered intravenously to a patient diagnosed with ovarian cancer to inhibit growth of the cancerous tumor. The dosing regimen is determined by methods known in the art considering the patient's clinical condition and the typical dosing regimen for topotecan as a free drug, namely 1.5 mg/m2 given as a 30 minute infusion over the course of 5 days in a 21 day cycle, repeated for 4 cycles. For example, a dosing regimen is 1.5 mg/m2 of the topotecan liposomes given as a 30 minute infusion over the course of 1-3 days in a week for 2 weeks in a 21 day cycle, repeated for 4 cycles.

What is claimed is:

1. A liposome comprising a conjugate bound to its lipid bilayer, the conjugate comprising:
(a) a vesicle-forming lipid having a polar head group and a hydrophobic tail, and
(b) a non-biological, biomimetic antagonist to a receptor upregulated at a disease site, directly or indirectly chemically linked to the polar head group of the vesicle-forming lipid.

2. A liposome according to claim 1 wherein the vesicle-forming lipid of the conjugate is selected from the group consisting of phospholipids, sterols, glycolipids, cationic lipids, sphingolipids, glycerolipids, hydrophilic polymer—derivatives of any of the foregoing lipids, and combinations thereof.

3. A liposome according to claim 1 wherein the vesicle-forming lipid of the conjugate is selected from the group consisting of gemini surfactants, phosphatidylethanolamines, phosphatidyl serines, sphingolipids, glycerolipids, hydrophilic polymer-derivatives of any of the foregoing lipids, and combinations thereof.

4. A liposome according to claim 1 wherein the vesicle-forming lipid of the conjugate is a hydrophilic polymer-derivative of a lipid selected from the group consisting of gemini surfactants, phosphatidylethanolamines, phosphatidyl serines, sphingolipids, and glycerolipids.

5. A liposome according to claim 1 wherein the vesicle-forming lipid of the conjugate is a hydrophilic polymer-derivative of a phosphatidylethanolamine or a gemini surfactant.

6. A liposome according to any of claims 2-5 wherein the hydrophilic polymer is selected from polyalkylethers, alkoxy-capped analogs of polyalkylethers, poly(sialic) acids, and analogs of poly(sialic) acids.

7. A liposome according to claim 6 wherein the hydrophilic polymer is polyoxyethylene glycol.

8. A liposome according to any of the preceding claims wherein the non-biological, biomimetic antagonist is an antagonist to a receptor upregulated in the vascular endothelium of inflammation, infection or tumor sites.

9. A liposome according to any of the preceding claims wherein the non-biological, biomimetic antagonist is an antagonist to a receptor selected from the group consisting of integrin receptors, Prostate Specific Membrane Antigen (PSMA) receptor, Herceptin, Tiel receptor, Tie2 receptor, ICAM1, Folate receptor, basic Fibroblast Growth Factor (bFGF) receptor, Epidermal Growth Factor (EGF) receptor, Vascular Endothelial Growth Factor (VEGF), Platelet Derived Growth Factor (PDGF) receptor, Laminin receptor, Endoglin, Vascular Cell Adhesion Molecule VCAM-1, E-Selectin, and P-Selectin.

10. A liposome according to claim 9 wherein the non-biological, biomimetic antagonist is an antagonist to an integrin receptor selected from the group consisting of αVβ3, αVβ55 and α5β1.

11. A liposome according to claim 10 wherein the non-biological, biomimetic antagonist is a vitronectin receptor (αVβ3) antagonist.

12. A liposome according to claim 11 wherein the vitronectin receptor antagonist is selected from compounds having the formula (I), (II), (III), (IV), (V), or (VI): 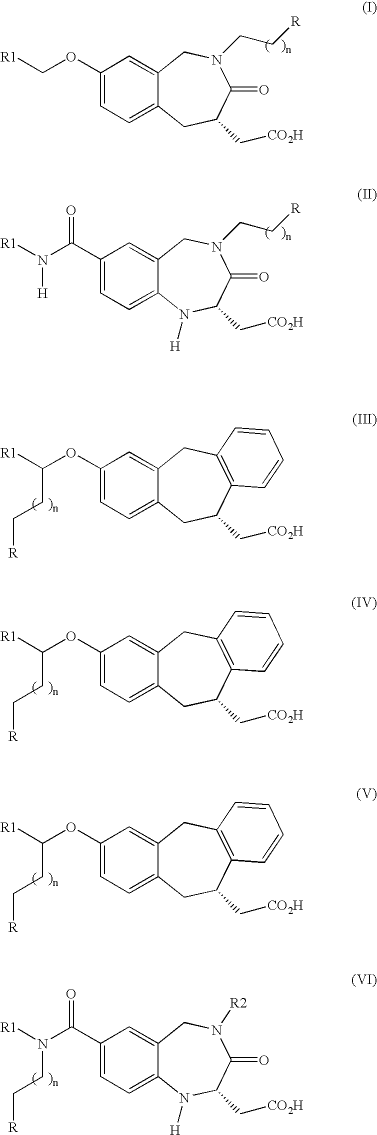
wherein the structures (I)-(VI):
R is selected from NH₂, COOH, and SH
R1 is selected from: 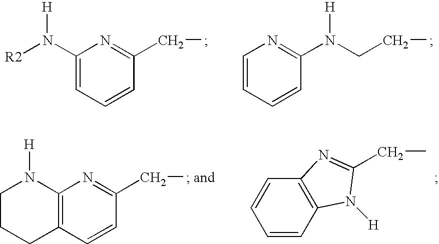
R2 is H or 1-4 C alkyl, and
n is an integer from 0-20.

13. A liposome according to claim 11 wherein the vitronectin receptor antagonist has the formula:

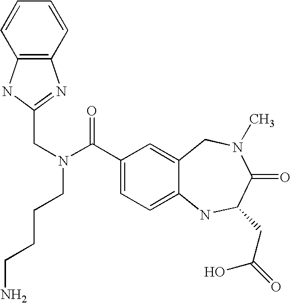

14. A liposome according to any of the preceding claims, further comprising a vesicle-forming lipid selected from the group consisting of phosphatidylcholines, sphingomyelin, and combinations thereof.

15. A liposome according to claim 14, wherein the phosphatidyl choline is selected from HSPC, DSPC, DPPC, DMPC, POPC, EggPC and combinations thereof.

16. A liposome according to claim 14 or 15, further comprising cholesterol.

17. A liposome according to any of claims 14, 15 or 16, further comprising a PEGylated lipid.

18. A liposome according to claim 1 substantially as hereinbefore defined with reference to Example 3.

19. A liposome according to any of the preceding claims, wherein the conjugate is inserted into the liposomal bilayer during formation of the bilayer.

20. A liposome according to any of the preceding claims wherein the liposome comprises a therapeutic active or a contrast agent suitable for diagnostic imaging entrapped in the liposome.

21. A conjugate useful for preparing a targeted liposome, comprising:
(a) a vesicle-forming lipid having a polar head group and a hydrophobic tail, and
(b) a non-biological, biomimetic antagonist to a receptor upregulated at a disease site, directly or indirectly chemically linked to the polar head group of the vesicle-forming lipid.

22. A conjugate according to claim 21 substantially as hereinbefore defined with reference to Example 2.

23. A method of treating or diagnosing a disease characterized by upregulation of a receptor, comprising administering to a patient in need thereof a safe and effective amount of a liposome according to any of claims 1-20, wherein the antagonist has binding affinity to the upregulated receptor.

24. A method according to claim 23 wherein the receptor is upregulated in the vascular endothelium of inflammation, infection or tumor sites.

25. A method according to claim 23 wherein the receptor is an integrin.

26. A method according to claim 23 wherein the receptor is the vitronectin receptor.

27. A method according to claim 23 wherein the disease is characterized by angiogenesis.

28. A method according to claim 23 wherein the disease is restenosis, osteo arthritis, rhumatoid arthritis, diabetic retinopathy, hemangiomas, psoriasis, or a cancerous tumor.

29. A pharmaceutical composition comprising the liposome according to any of claims 1-20 and a pharmaceutically acceptable carrier or diluent.

30. Use of a liposome according to any of claims 1-20 in the manufacture of a medicament for use in the treatment of a disease characterized by upregulation of the receptor.

31. A liposome according to any of claims 1-20 for use in treating a disease characterized by upregulation of the receptor.
receptor, ICAMI, Folate receptor, basic Fibroblast Growth Factor receptor, Epidermal Growth Factor receptor, Vascular Endothelial Growth Factor, Platelet Derived Growth Factor receptor, Laminin receptor, Endoglin, Vascular Cell Adhesion Molecule VCAM-1, E-Selectin, and P-Selectin.

14. (Amended) A liposome according to claim 1, further comprising a vesicle-forming lipid selected from the group consisting of phosphatidylcholines, sphingomyelin, and combinations thereof.

16. (Amended) A liposome according to claim 14, further comprising cholesterol.

17. (Amended) A liposome according to claim 14, further comprising a PEGylated lipid.

19. (Amended) A liposome according to claim 1, wherein the conjugate is inserted into the liposomal bilayer during formation of the bilayer.

20. (Amended) A liposome according to claim 1 wherein the liposome comprises a therapeutic active or a contrast agent suitable for diagnostic imaging entrapped in the liposome.

23. (Amended) A method of treating or diagnosing a disease characterized by upregulation of a receptor, comprising administering to a patient in need thereof a safe and effective amount of a liposome according to claim 1, wherein the antagonist has binding affinity to the upregulated receptor.

29. (Amended) A pharmaceutical composition comprising the liposome according to claim 1 and a pharmaceutically acceptable carrier or diluent.
